(19) United States
(12) Patent Application Publication
(10) Pub. No.: US 2004/0176294 A1
(43) Pub. Date: Sep. 9, 2004

(54) Use of thyroid-stimulating hormone to induce lipolysis

(76) Inventor: James D. Kelly, Mercer Island, WA (US)

Correspondence Address: ZymoGenetics, Inc., 1201 Eastlake Avenue East, Seattle, WA, 98102, US

(21) Appl. No.: 10/794,155

(22) Filed: Mar. 5, 2004

Related U.S. Application Data
(60) Provisional application No. 60/451,966, filed on Mar. 5, 2003.

(51) Int. Cl. A61K 38/23

(52) U.S. Cl. 514/12

(57) ABSTRACT

The use of thyroid-stimulating hormone (TSH) to induce lipolysis, treat obesity, insulin resistance, liver steatosis, hyperlipidemia, and type-2 diabetes is described.

### REFERENCE TO RELATED APPLICATIONS

This application claims the benefit of U.S. Provisional Application Serial No. 60/451,966, filed Mar. 5, 2003, which is herein incorporated by reference.

### FIELD OF THE INVENTION

The present invention relates to the treatment of obesity, the complications associated with obesity, liver steatosis, insulin resistance, and diabetes. More particularly, the invention relates to the use of thyroid-stimulating hormone (TSH or thyrotropin) to stimulate lipolysis for the treatment of obesity, complications associated with obesity, liver steatosis, insulin resistance, and diabetes.

### BACKGROUND OF THE INVENTION

Obesity is a public health problem that is both serious and widespread. One third of the population in industrialized countries has an excess weight of at least 20% relative to the ideal weight. This phenomenon has spread to the developing world, particularly to the regions of the globe where economies are modernizing. As of the year 2000, there were an estimated 300 million obese people worldwide.

Obesity considerably increases the risk of developing cardiovascular or metabolic diseases. For an excess weight greater than 30%, the incidence of coronary diseases is doubled in subjects less than 50 years of age. Studies carried out for other diseases are equally revealing. For an excess weight of 20%, the risk of high blood pressure is doubled. For an excess weight of 30%, the risk of developing non-insulin dependent diabetes is tripled, and the incidence of dyslipidemia increased six fold. The list of additional diseases promoted by obesity is long; abnormalities in hepatic function, digestive pathologies, certain cancers, and psychological disorders are prominent among them.

Treatments for obesity include restriction of caloric intake, and increased caloric expenditure through physical exercise. However, the treatment of obesity by dieting, although effective in the short-term, suffers from an extremely high rate of recidivism. Treatment with exercise has been shown to be relatively ineffective when applied in the absence of dieting. Other treatments include gastrointestinal surgery or agents that limit the absorption of dietary lipids. These strategies have been largely unsuccessful due to side effects of their use.

Current therapies for complications associated with obesity, including type-2 diabetes, hyperlidpemia, and steatohepatitis, have been inadequate to halt the progression of these life-threatening pathologies in most instances.

Lipolytic agents have been investigated extensively and found to produce striking improvements in adiposity, glucose sensitivity, and dyslipidemic conditions. These agents, agonists of sympathetic nervous system catecholamines have not proven to be successful therapeutics principally due to the inability thus far to create specific agents that target only adipose tissue without stimulating other tissues responsive to sympathetic innervation.

Clearly there remains a need for novel treatments that are useful for reducing body weight and the deleterious effects associated with increased adiposity in humans. Therapies that can be administered to promote lipolysis and weight loss would help to control obesity and thereby alleviate many of the negative consequences associated with this condition.

### BRIEF DESCRIPTION OF THE DRAWINGS

FIG. 1. Dose response of TSH and isoproterenol-induced lipolysis in 3T3 L1 adipocytes. Glycerol (upper panel) and free fatty acid (FFA; lower panel) accumulations were determined following a 4-hour treatment with TSH (solid squares) or isoproterenol (solid triangles) at the indicated concentrations.

FIG. 2. Stimulation of lipolysis in vivo by TSH. Male ob/ob mice (n=7-8/group) were injected with vehicle saline, TSH (300 μg/kg), or the P₃-AR agonist CL 316,243 (1 mg/kg). Changes from baseline in serum glycerol (upper panel) and FFA (lower panel) at 2 and 4 hours post-injection were determined for each group as described in Example 3. Error bars are standard error of measurement.

FIG. 3. Thyroid hormone levels in male ob/ob mice following 25 days of treatment with TSH. Mice (n=7-8/group) were injected daily with vehicle saline, TSH (267 μg/kg), P₃-AR agonist CL 316,243 (1 mg/kg), or thyroxine (1-1.5 μg/mouse; see example 4). The level of total circulating T₄ in serum was determined by ELISA.

FIG. 4. Serum glucose levels in male ob/ob mice following 28 days of treatment with TSH. Animals (n=4/group) were fasted for 4 hours immediately following the dark cycle, then blood was drawn, serum separated, and glucose levels determined by enzymatic methods. Treatment groups are as described in FIG. 3 and symbols for each group are shown in the figure legend.

FIG. 5. Glucose challenge of animal groups described in FIG. 4. Following blood sampling to measure basal glucose and insulin levels, glucose (1.5 g/kg), was injected at time zero and blood sampled again at 20, 40, and 120 minutes following injection. Panel A depicts blood glucose levels and Panel B, serum insulin levels.

### SUMMARY OF THE INVENTION

In one aspect, the invention provides a method for inducing lipolysis in a mammal comprising administering to the mammal a pharmaceutically effective amount of a TSH polypeptide, wherein administration of the polypeptide results in a clinically significant decrease in the body weight of the mammal. In an embodiment, the mammal is obese. In another embodiment, the mammal has a body mass index greater than 25. In a further embodiment, the body mass index is 26, between 26 and 50, or greater than 50. In another embodiment, the decrease in body weight results fro lipolytic stimulation of adipose tissue.

In another aspect, the invention provides, a method for inducing weight loss in a mammal, comprising administering to the mammal a pharmaceutically effective amount of a TSH polypeptide, wherein administration of the polypeptide results in a clinically significant decrease in body weight of the mammal. In an embodiment, the mammal is obese. In another embodiment, the mammal has a body mass index greater than 25. In a further embodiment, the body mass index is is 26, between 26 and 50, or greater than 50.

In another aspect, the invention provides, a method for improving insulin sensitivity in a mammal comprising administering to the mammal a pharmaceutically effective amount of a TSH polypeptide, wherein administration of the polypeptide results in increased sensitivity to insulin. In an embodiment, the blood glucose levels in the mammals are decreased. In another embodiment, the insulin levels are decreased.

In another aspect, the invention provides, a method for treating, or a method for preventing type-2 diabetes in a mammal, comprising administering to the mammal a pharmaceutically effective amount of a TSH polypeptide, wherein administration of the polypeptide results in an improvement in the diabetic state of the mammal. In an embodiment the mammal is obese. In another embodiment, administration of the polypeptide results in deceased serum glucose and/or serum insulin levels.

In another aspect, the invention provides, a method for treating hyperlipidemia in a mammal comprising administering to the mammal a pharmaceutically effective amount of a TSH polypeptide, wherein administration of the polypeptide results in decreased hyperlipidemia in the mammal. In an embodiment, the mammal is obese. In another embodiment, the mammal is type-2 diabetic. In another embodiment, the serum cholesterol and/or triglyceride levels of the mammal are decreased.

In another aspect, the invention provides, a method for treating steatohepatitis in a mammal, comprising administering to the mammal a pharmaceutically effective amount of a TSH polypeptide, wherein administration of the polypeptide results in an improved steatohepatic state in the mammal. In an embodiment, the mammal is obese. In another embodiment, the mammal is type-2 diabetic.

In another aspect, the invention provides, a method for preventing steatohepatitis in a mammal with steatosis, comprising administering to the mammal a pharmaceutically effective amount of a TSH polypeptide, wherein administration of the polypeptide maintains or reduces the steatosis. In an embodiment, the mammal is obese. In another embodiment, the mammal is type-2 diabetic.

In another aspect, the invention provides, a method for lowering elevated plasma cholesterol in a mammal, comprising administering a pharmaceutically effective amount of a TSH polypeptide to said mammal, wherein administration of the polypeptide lowers the plasma cholesterol level in the mammal. In an embodiment, the mammal is type-2 diabetic and/or obese. In another embodiment, the mammal is hypercholesterolemic.

In another aspect, the invention provides a method of lowering elevated triglyceride levels in a mammal, comprising administering a pharmaceutically effective amount of a TSH polypeptide to said mammal, wherein administration of the polypeptide lowers triglyceride levels in the mammal. In an embodiment, the mammal is type-2 diabetic. In another embodiment, the mammal is hypertriglyceridemic.

In another aspect, the invention provides a method for treating steatosis of the liver in a mammal with steatosis, comprising administering to the mammal a pharmaceutically effective amount of a TSH polypeptide, wherein administration of the polypeptide maintains or reduces the steatosis. In an embodiment, the mammal is obese. In another embodiment, the mammal is type-2 diabetic.

In another aspect, the invention provides a method for treating atherosclerosis, comprising comprising administering to the mammal a pharmaceutically effective amount of a TSH polypeptide, wherein administration of the polypeptide improves the athersclotic state. In an embodiment, the mammal is hyperlipidemic.

These and other aspects of the invention will become evident upon reference to the following detailed description of the invention.

### DETAILED DESCRIPTION OF THE INVENTION

The present invention fills the need for a novel therapy to promote weight loss and/or treat the diabetic state frequently associated with obesity. The present invention comprises administering thyroid stimulating hormone (TSH) to an individual to promote lipolysis and thereby promote weight loss, reduce liver steatosis, and/or increase insulin sensitivity. The present invention further comprises a method for treating type-2 diabetes or a pre-diabetic condition in an individual comprising administering TSH to said individual. The invention further comprises a method for treating type-2 diabetes or a pre-diabetic condition in an individual comprising administering a pharmaceutically effective amount of TSH to the individual. Additionally, the present invention comprises a method for improving insulin sensitivity in an individual comprising administering TSH to said individual without disruption of the thyroid axis. In an aspect, the individual is treated with a therapeutically effective amount of TSH. In another aspect, TSH is used to promote reversal of steatosis or steatohepatitis. In an aspect of the invention the individual is a mammal. In an embodiment the mammal is human.

The teachings of all of the references cited herein are incorporated in their entirety herein by reference.

Herein we disclose methods that are useful for the treatment of obesity. As described below, the ability to stimulate lipolysis in adipose tissue provides a means of intervening in a wide number of pathologies associated with obesity. In particular, we have discovered that TSH, when administered in vitro or in vivo, potently stimulates lipolysis. As a consequence, metabolic rate is increased, leading to decreased weight, increased insulin sensitivity, and decreased serum hyperlipidemia. This increase in metabolism is independent of the activation of the thyroid axis. Further, we have discovered a method of administration of TSH that stimulates lipolysis directly, without chronic elevation of thyroid hormone levels.

When used to promote lipolysis, TSH can promote weight loss. The invented methods are useful for treating conditions that include: obesity, atherosclerosis associated with obesity or dyslipidemia, diabetes, hypertension associated with obesity or diabetes, steatosis or steatohepatitis, or more generally the various pathologies associated with obesity.

In another aspect of the invention, TSH can be used for the maintenance of weight loss in individuals who are treated with other medicaments that induce weight loss.

The invention is also useful for the treatment of non-insulin dependent diabetes, especially that associated with obesity. In one embodiment, the use of TSH to treat non-insulin dependent diabetes is envisioned in non-obese individuals.

The invention is further useful for the treatment of dyslipidemias, including hypercholesterolemia and hypertrglyceridemia.

Yet another aspect of the invention relates to the use of TSH to increase resting metabolic rate in individuals. In one embodiment, individuals with low resting metabolic rate are administered TSH to promote lipolysis and increase energy utilization while maintaining a euthyroid state.

Energy expenditure represents one side of the energy balance equation. In order to maintain stable weight, energy expenditure should be in equilibrium with energy intake. Considerable efforts have been made to manipulate energy intake (i.e., diet and appetite) as a means of maintaining or losing weight; however, despite enormous sums of money devoted to these approaches, they have been largely unsuccessful. There have also been efforts to increase energy expenditure pharmacologically as a means of managing weight control and treating obesity. Increasing energy metabolism is an attractive therapeutic approach because it has the potential of allowing affected individuals to maintain food intake at normal levels. Further, there is evidence to support the view that increases in energy expenditure due to pharmacological means are not fully counteracted by corresponding increases in energy intake and appetite. See Bray, G. A. (1991) Ann Rev Med 42, 205-216.

Energy expenditure can be stimulated pharmacologically by manipulation of the central nervous system, by activation of the peripheral efferents of the sympathetic nervous system (SNS), or by increasing thyroid hormone levels.

Thyroid hormone stimulates carbohydrate and lipid catabolism in most cells of the body and increases the rate of protein synthesis. TSH stimulates thyroid hormone biosynthesis and secretion. The secretion of TSH from the thyrotrophs of the anterior pituitary is inhibited by circulating T₄ and T₃ and stimulated by thyrotropin-releasing hormone produced in the hypothalamus See Utiger, in Endocrinology and Metabolism (Felig and Frohman, eds), pp. 261-347, McGraw-Hill, (2001).

As a result of the catabolism produced by thyroid hormone, heat is given off and energy expenditure is increased. There has been an intense interest in thyroid hormone levels in obesity, due to the opportunity to increase basal energy consumption by increasing thyroid hormone levels. Studies have revealed that obese and normal-weight individuals have similar thyroid hormone profiles. An excess of thyroid hormone leads to various disorders, generally termed thyrotoxicosis. This condition is characterized by an abnormally high metabolic rate, increased blood pressure, high body temperature, heat intolerance, irritability, and tremors of the fingers. Of particular concern in the obese state is the tendency to increased and more forceful heartbeats.

Due to the adverse effects of elevated thyroid hormone levels, the use of thyroid hormone to treat obesity has seen little success, other than in the small fraction of obese patients identified with hypothyroidism.

Much of the energy expended on a daily basis derives from resting metabolic rate (RMR), which comprises 50-80% of the total daily energy expenditure. For a review, see Astrup, A. (2000) Endocrine 13, 207-212. Noradrenaline turnover studies have shown that most of the variability in RMR that is unexplained by body size and composition is related to differences in SNS activity, suggesting that SNS activity does modulate RMR. See Snitker, S., et al. (2001) Obes. Rev. 1, 5-15. Meal ingestion is accompanied by increased SNS activity, and studies have demonstrated that increased SNS activity in response to a meal accounts for at least part of meal-induced thermogenesis.

The peripheral targets of the SNS involved in the regulation of energy utilization are the β-adrenoreceptors (β-AR's). These receptors are coupled to the second messenger cyclic adenosine monophosphate (cAMP). Elevation of cAMP levels leads to activation of protein kinase A (PKA), a multi-potent protein kinase and transcription factor eliciting diverse cellular effects. See Boume, H. R., et al. (1991) Nature 349, 117-127. Adipose tissue is highly innervated by the SNS, and possesses three known subtypes of β-adrenoreceptors, β₁-, β₂-, and β₃-AR. Activation of the SNS stimulates energy expenditure via coupling of these receptors to lipolysis and fat oxidation. Increased serum free fatty acids (FFAs) produced by adipose tissue and released into the bloodstream stimulate energy expenditure and increase thermogenesis. For a review, see Astrup, A. (2000) Endocrine 13, 207-212. In addition, elevated PKA levels increase energy utilization in fat by up-regulating uncoupling protein-1 (UCP-1), which creates a futile cycle in mitochondria, generating waste heat.

Over the past two decades, investigation of the physiological benefits of SNS activation for the treatment of obesity and diabetes related to obesity has centered on pharmacological activation of the P₃-AR. Expression of the P₃-AR is restricted to a narrower range of tissues than the β₁ or β₂ isoforms, and is highly expressed in rodent adipose tissue compared to the other isoforms. Experimental work in rodents treated with P₃-AR agonists has demonstrated that stimulation of lipolysis and fat oxidation produces increased energy expenditure, weight loss, and increased insulin sensitivity. See de Souza, C. J. and Burkey, B. F. (2001) Curr Pharm Des 7, 1433-1449. However, the potential benefits of the P₃-AR agonists have not been realized, due to their lack of efficacy at the human P₃-AR. Further, it has more recently been shown that the levels of P₃-AR in rodent adipose tissue are much higher than in human adipose tissue. In human adipose tissue, the β₁ and β₂ isoforms represent the predominant adrenoreceptor isoforms. See Arch, J. R. (2002) Eur J Pharmacol 440, 99-107. Thus, although the proof-of-concept of stimulation of lipolysis for treatment of obesity has been clearly demonstrated in rodents, the mechanism for therapeutically producing the corresponding effects in humans is unrealized.

Strategies to promote lipid oxidation through lipolysis have demonstrated improved insulin sensitivity at doses that do not promote weight loss, and over time periods that do not affect body weight. An insulin-sensitizing effect is more readily detectable than an anti-obesity effect. Stimulation of fat oxidation may rapidly lower the intracellular concentration of metabolites that modulate insulin signaling; the anti-obesity effect, in contrast, must develop gradually as large stores of fat are oxidized.

The present invention relates generally to methods that are useful for stimulating lipolysis in adipose cells and/or tissue. Those having ordinary skill in the art will understand that lipolysis is the biochemical process by which stored fats in the form of triglycerides are released from fat cells as individual free fatty acids into the circulation. Stimulation of lipolysis has been clearly linked to increased energy expenditure in humans, and several strategies to promote lipolysis and increase oxidation of lipids have been investigated to promote weight loss and treat the diabetic state associated with obesity. These therapeutic efforts primarily focus on creating compounds that stimulate the sympathetic nervous system (SNS) through its peripheral β-adrenoreceptors. The discovery of potent TSH-promoted lipolysis in adipose tissue, both in vivo and in vitro, presents a novel and specific method of treating obesity, as well as the insulin-resistant diabetic state associated with obesity.

As used herein, the term “brown fat” refers to adipose tissue depots that contain high densities of mitochondria, and whose primary function is the production of heat through uncoupling of fat oxidation to ATP generation in the mitochondria, (a “futile” cycle). The term “white fat” refers to the predominant form of adipose tissue and serves as the principal storage depot for fatty acids in the form of triglycerides.

As used herein, the terms “obesity” and “obesity-related” are used to refer to individuals having a body mass which is measurably greater than ideal for their height and frame. For example, these terms refer to individuals with body mass index values of greater than 25, equal to or greater than 30, equal to or greater than 35, and greater than 40.

Thyroid-stimulating hormone (TSH), is a ˜30 kDa glycoprotein composed of two non-covalently linked peptide subunits, an alpha subunit and a beta subunit. The alpha subunit of TSH is the same as that of luteinizing hormone, follicle-stimulating hormone, and chorionic gonadotropin, and has the amino acid sequence of: mdyyrkyaaiflvtlsvflhylhsapdvqdcpectlqenpffsqpgapilqcmgccfsrayptplrskktmlvqknvtses tccvaksynrvtvmggfkvenhtachcstcyyhks (SEQ ID NO: 1). A polynucleotide sequence for SEQ ID NO:1 is shown in SEQ ID NO:2. Amino acids 25 to 116 of the alpha subunit comprise the mature protein (SEQ ID NO:3). The beta subunit of TSH is unique, and has the amino acid sequence of: mtalflmsmlfglacgqamsfcipteytmhierrecaycltintticagycmtrdingklflpkyalsqd vctyrdfiyrtveipgcplhvapyfsypvalsckcgkcntdysdciheaiktnyctkpqksylvgfsv (SEQ ID NO: 4) and determines the hormone's biological specificity. A polynucleotide sequence for SEQ ID NO:4 is shown in SEQ ID NO:5. Amino acids 21 to 132 of the beta subunit comprise the mature protein (SEQ ID NO:6). TSH may be produced by biopharmaceutical methods using skills recognized in the art, or may be obtained from commercial sources, such as, for example, Genzyme Corporation (Cambridge, Mass.). The thyroid gland produces two thyroid hormones, thyroxine (T₄) and triiodothyronine (T₃). The ratio of T₄ to T₃ in normal human serum is typically 100:1. Total thyroid hormone levels in a normal human range from 5-11 μg/dl of serum; this range is defined as the euthyroid state. Excess levels of thyroid hormones (thyrotoxicosis) result in a hyperthyroid condition, and low levels of thyroid hormones in serum are defined as a hypothyroid state.

Steatosis is the accumulation of fat deposits in the liver. Steatosis of any etiology can be associated with the development of fibrosis, so called steatohepatitis, and even cirrhosis of the liver.

TSH Promotes Elevation of cAMP in Adipose Tissue

TSH exerts its effects through interaction with the thyroid-stimulating hormone (TSH) receptor. See Nakabayashi, K., et al. (2002) J Clin Invest 109, 1445-1452. The TSH receptor (TSHR) is a member of the G-protein coupled, seven-transmembrane receptor superfamily. Activation of the TSH receptor leads to coupling with heterotrimeric G proteins, which evoke downstream cellular effects. The TSH receptor has been shown to interact with G proteins of subtypes Gₛ, G_{q}, G₁₂, and Gᵢ. In particular, interaction with Gₛ, leads to activation of adenyl cyclase and increased levels of cAMP. See Laugwitz, K. L., et al. (1996) Proc Natl Acad Sci USA 93, 116-120.

Recent reports have documented the presence of TSHR in adipose tissue of humans and rodents. See Bell, A., et al. (2000) Am J Physiol Cell Physiol 279, C335-340, and Endo, T., et al. (1995) J Biol Chem 270, 10833-10837.

Example 1 demonstrates the production of elevated cAMP by TSH in cultured murine 3T3-L1 adipocytes and in primary human adipocytes. We have discovered that TSH produces activation of a luciferase reporter gene construct under the control of cAMP response element (CRE) enhancer sequences. We typically observe a 10- to 40-fold induction of the luciferase reporter gene in response to TSH treatment, indicating significant production of cAMP in adipocytes following activation of the TSHR. Thus, TSH could be an important physiological regulator of adipose tissue lipolysis, which is primarily controlled by intracellular cAMP levels. For a review, see Astrup, A. (2000) Endocrine 13, 207-212.

TSH Promotes Lipolysis in Adipocytes and Whole Animals

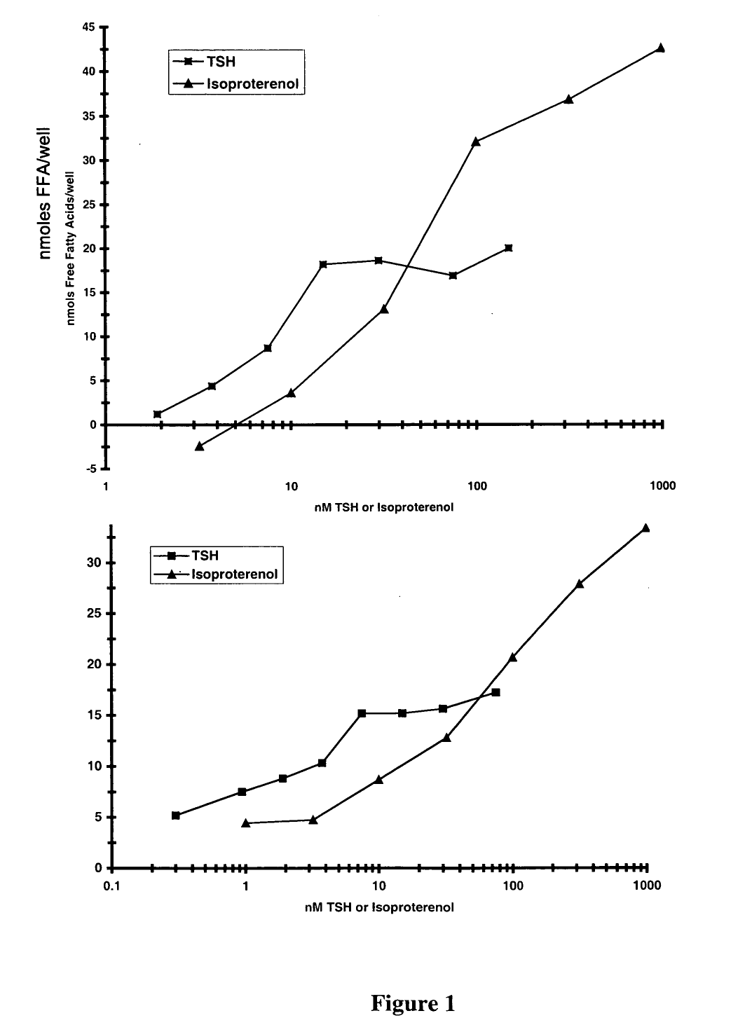

TSH was examined for its ability to activate lipolysis in cultured murine 3T3-L1 adipocytes as described in Example 2. Following treatment of adipocytes with test compounds for 4 hours, lipolysis was assessed by the accumulation of glycerol and free fatty acid (FFA) in the adipocyte culture medium. Treatment of adipocytes with 10 nM recombinant human TSH produced near maximal levels of extracellular glycerol and FFA. FIG. 1 compares the lipolytic activity of TSH to isoproterenol, a non-specific α-adrenergic agonist. Maximal lipolysis achieved with TSH is approximately 50% of that produced by isoproterenol. Lipolysis is significantly stimulated by TSH at concentrations of 1 nM, indicating that TSH is a potent regulator of lipolysis in adipocytes.

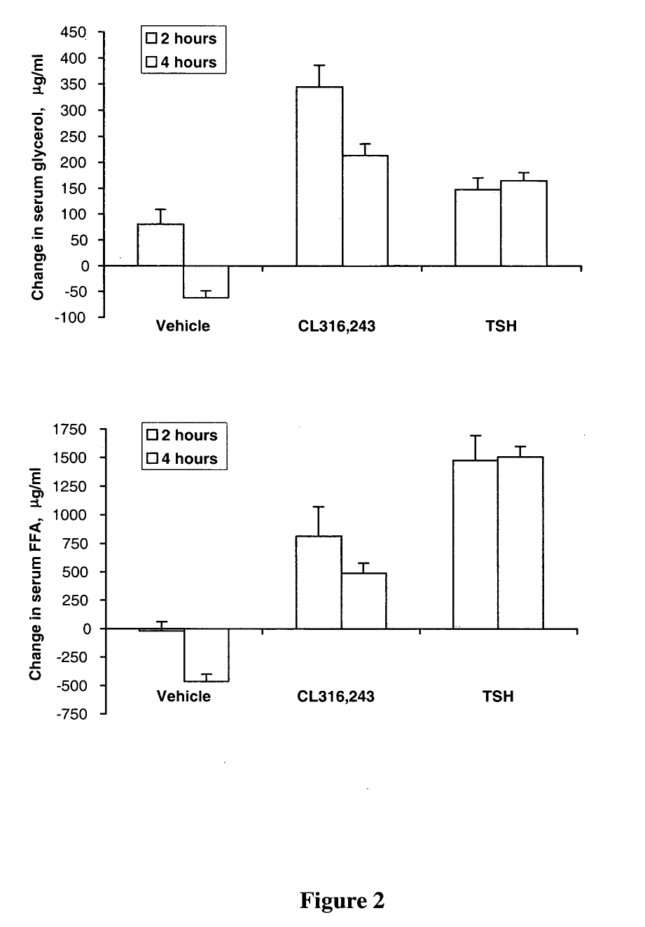

A significant aspect of the invention is the stimulation of lipolysis in vivo. Intraperitoneal (IP) injection of TSH produces acute elevation of serum glycerol and FFA in whole animals. As described in Example 3, mice were fasted overnight before IP injection of TSH (300 μg/kg), P₃-AR agonist CL 316,243 (1 mg/kg), or vehicle saline. Serum was sampled before injection to establish baselines, then sampled again at 2 and 4 hours post-injection. Although the vehicle controls show decreases in serum glycerol and FFA levels by four hours, the animals treated with TSH show significant elevations in both, indicating that TSH is a potent stimulator of lipolysis in vivo. As demonstrated in FIG. 2, the invention is a potent stimulator of increased serum FFA in vivo, showing significant increases over the P₃-AR agonist at the 4-hour time point. In one embodiment of the invention, TSH is used to produce acute increases in plasma FFA, thus promoting increased basal metabolic rate.

Chronic Treatment of ob/ob Mice with TSH Produces Lipolysis Without Sustained Increases in Thyroid Hormone Levels

Obese ob/ob mice are frequently used as models of human obesity and diabetes. To examine the effects of TSH-stimulated lipolysis in this model, TSH, β₃-AR agonist CL 316,243, thyroxine, or vehicle saline were administered daily for 4 weeks by IP injection as described in Example 4. A thyroxine group was included to distinguish the metabolic effects of thyroid hormone from the direct stimulation of lipolysis in adipocytes mediated by TSH.

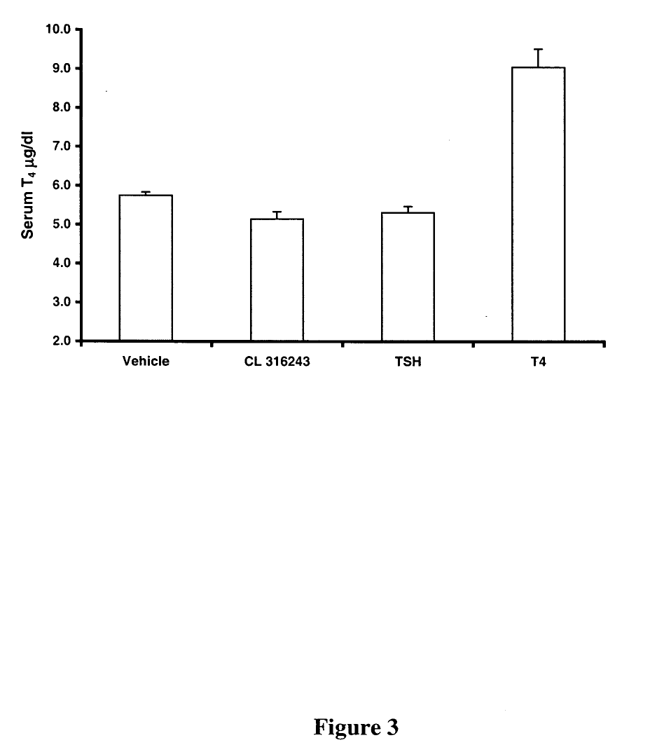

An aspect of the instant invention is the discovery that TSH, when introduced into the periphery by IP injection at doses that stimulate lipolysis, does not result in the creation of a chronic hyperthyroid state. As shown in FIG. 3, circulating levels of thyroid hormone (T₄) following one month of daily injections of TSH do not increase above the levels found in the vehicle controls. The TSH amounts injected daily are greater than 100 times the amount of TSH that would be expected to be released in a single day from the pituitary. The present invention thus provides for a method of introducing TSH without altering the thyroid hormone axis to produce a profound hyperthyroid state, while stimulating lipolysis to produce a therapeutic effect for the treatment of obesity and diabetes.

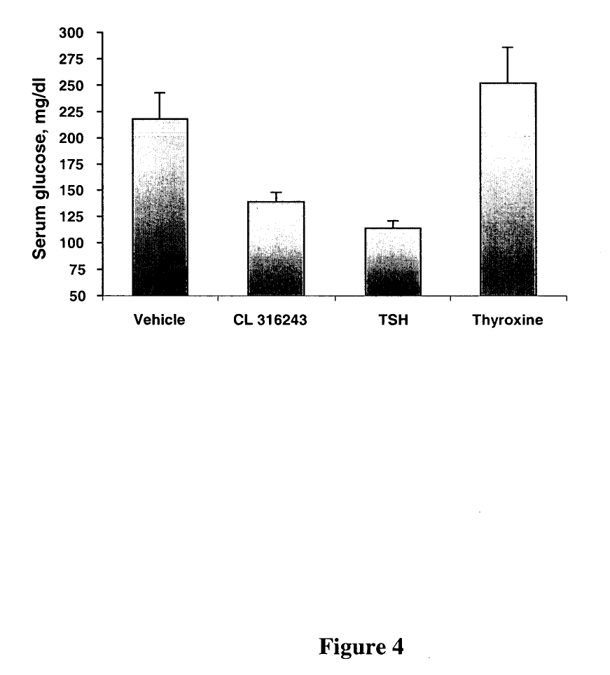

Following four weeks of treatment, injected animals were subjected to an intraperitoneal glucose tolerance test (IPGTT) in order to evaluate the effect of TSH treatment on serum glucose levels and insulin sensitivity. The subject mice were fasted for four hours before blood sampling to obtain baseline glucose and insulin levels, then were injected with glucose to allow for measurement of insulin sensitivity to a glucose challenge. As shown in FIG. 4, the fasted serum glucose levels in the TSH-treated animals were significantly lower than in the vehicle controls or the thyroxine-treated animals after 4 weeks of treatment. TSH is seen to be as effective as the control P₃-AR agonist in reducing hyperglycemia in this model. FIG. 4 demonstrates the discovery that peripheral administration of TSH does not act to reduce blood glucose via thyroid hormone activity, as increasing thyroid hormone levels by administration of thyroxine does not reduce fasting glucose levels compared to the vehicle control group.

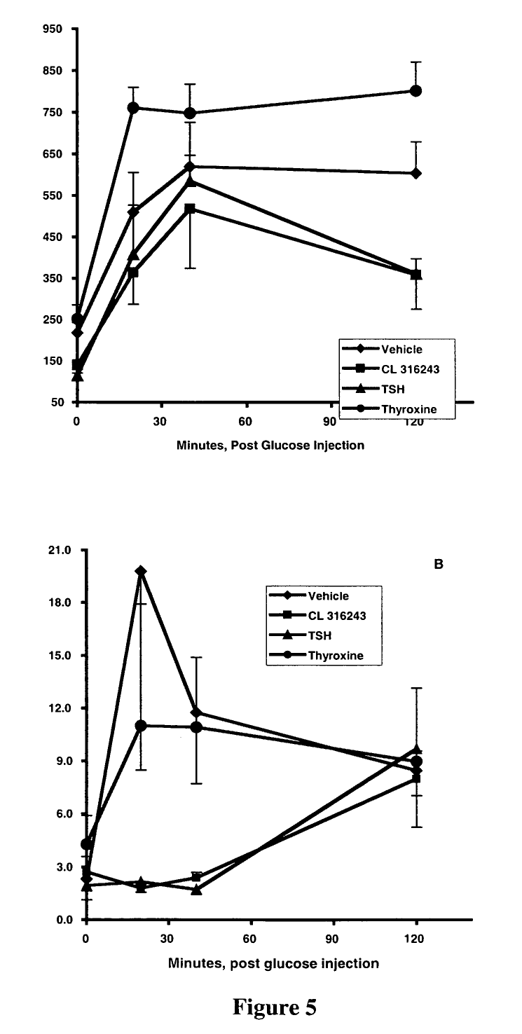

An additional aspect of the invention is the improvement of insulin sensitivity and reduced hyperglycemia in response to a glucose challenge, as shown in FIG. 5. The glucose tolerance test is a typical diagnostic measure of diabetes and insulin sensitivity. See Defronzo R. A. et al. (1991) Diabetes Care 14, 173-194. Panel A demonstrates that the clearance of a glucose challenge is significantly enhanced in the TSH-treated group compared to vehicle- or thyroxine-treated groups. Panel B shows that insulin sensitivity in the TSH-treated group is significantly improved compared to vehicle- or thyroxine-treated controls. The TSH and control P₃-AR agonist groups exhibit enhanced glucose disposal with lower insulin levels compared to the vehicle or thyroxine treatment groups.

In a further aspect of the invention, stimulation of lipolysis by TSH results in decreased serum lipid levels. Specifically, chronic treatment of ob/ob mice with TSH leads to significant reductions in serum cholesterol and triglyceride levels. The invention comprises a method for lowering elevated plasma cholesterol and triglyceride levels typically associated with obesity and type-2 diabetes. The discovery that TSH-stimulated lipolysis produces improvement in hyperlipidemia stands in contrast to the observation that sympathetic stimulation of lipolysis with β-AR agonists result in no reduction in serum cholesterol levels, and typically result in unchanged or slightly increased serum triglyceride levels (e.g., serum lipid analysis data in Example 4). Further, the effects of TSH on lowering serum triglyceride and cholesterol levels are not due to increases in the circulating levels of thyroid hormone. As detailed in Example 4, chronic treatment with thyroid hormone resulted in elevated plasma triglycerides and did not reduce serum cholesterol levels.

The invention further provides a method for the treatment of obesity. Stimulation of lipolysis can result in weight loss and reductions in fat mass in animals and humans. Increased lipolysis in fat through SNS stimulation by β-AR agonists typically results in increases in scapular brown fat mass in rodents, and decreases in white fat tissue mass. The increased brown fat mass results in a higher metabolic rate, due to the oxidation of lipids for the production of heat in brown fat. Mice treated with TSH had increased brown fat mass over vehicle controls (Example 4). Further, TSH-treated mice had significant decreases in the mass of mesangial intra-abdominal white fat. In addition, body weight increases in the TSH group were reduced compared to controls (p=0.11). The TSH treatment group (n=8) contained the only individuals (n=3) that exhibited decreases from starting body weight after one month of treatment. As described above, the anti-obesity effect of treatment with TSH is expected to develop more slowly than the insulin-sensitizing effect.

In a further embodiment of the invention, TSH is useful for the treatment of steatosis and steatohepatitis. Although liver disease is not a widely appreciated complication of obesity, epidemiologic evidence suggests that obesity increases the risk of cirrhosis. For example, in autopsy series, obesity was identified as the only risk factor for disease in 12% of cirrhotic subjects. See Yang, S. Q. et al. (1997) Proc Natl Acad Sci USA 94, 2557-2562. Notably, cirrhosis is approximately six times more prevalent in obese individuals than in the general population. In the USA, the high percentage of overweight people in the general population partially explains the fact that non-alcoholic fatty liver disease (NAFLD) is the most common liver disease. Type-2 diabetes is present in 33% of these subjects. The degree of obesity correlates positively with the prevalence and severity of fatty liver (steatosis), and this in turn correlates with steatohepatitis. A current explanation of the pathogenesis of steatohepatitis is the “two-hits” hypothesis. See Day, C. P, and James, O., Gastroenterology 114, 842-845. The first “hit” is the depositing of fat in hepatocytes, leading to fatty degeneration of the liver or steatosis hepatitis. This fatty degeneration increases the organ's sensitivity to the second “hit”, which can be any one of a variety of insults including diabetes, lipid peroxidation due to drug metabolism, or excess alcohol intake.

As detailed in Example 4, chronic treatment of ob/ob mice with TSH significantly reverses steatosis in these subjects. The instant invention thus produces a method for reversing the first “hit” thought to be required for the progression to steatohepatitis and cirrhosis. Further, treatment with the invention of those with steatohepatitis, for whom no efficacious therapy is currently available, may induce a reversion to a normal (non-steatotic) hepatic state, preventing the progression of pre-cirrhotic hepatitis to cirrhosis.

Advantages of TSH as a Lipolysis-Stimulating Agent

The invention comprises a novel method of producing lipolysis and increasing metabolic rate. Other strategies for therapeutically inducing lipolysis employed thus far have suffered from a lack of specificity, such as P-AR agonists in general, or a lack of efficacy, as for the most specific of the β₃-AR agonists developed to date. Most of the agents investigated for human use have not exhibited sufficient selectivity, and as a result have produced increased blood pressure and heart rate due to activation of sympathetic pathways in tissues other than adipose. See Arch, J. R. (2002) Eur J Pharmacol 440, 99-107.

In spite of the emphasis on development of P₃-AR-specific agonists, recent human studies have implicated the β₁- and β₂-adrenoreceptors as the primary mediators of sympathetically induced thermogenesis and energy expenditure. Further, studies in human obese populations suggest that the decreases in resting metabolic rate observed in these individuals are the result of impaired function of β₂-adrenoreceptors in adipose tissue. See Schiffelers, S. L., et al. (2001) J Clin Endocrinol Metab 86, 2191-2199, and Blaak, E. E., et al. (1993) Am J Physiol 264, E11-17. Thus, a novel mechanism of increasing lipolysis without invoking sympathetic innervation presents a unique opportunity for the treatment of obesity.

Other studies in human lean and obese subjects have found that increases in plasma FFA levels lead to increases in lipid oxidation and energy expenditure. These studies conclude that the accumulation of fat in obese subjects may be due to a defect in adipose tissue lipolysis rather than to defects in lipid utilization. See Schiffelers, S. L., et al. (2001) Int J Obes Relat Metab Disord 25, 33-38.

Increased lipolysis in adipose tissue and the resulting decrease in adipocyte size are negatively correlated with insulin resistance in human cross-sectional studies. See Weyer, C., et al. (2000) Diabetologia 43, 1498-1506. Thus a method for stimulating lipolysis and reducing adipocyte size is predicted to decrease the insulin-resistant diabetic state associated with obesity. The presence of significant numbers of TSH receptors in adipose tissue represents a novel method for the control of lipolysis and RMR in human obese populations.

Use of TSH to Treat Type-2 Diabetes

TSH can also be administered to treat type-2 diabetes mellitus (Type-2 DM). Type-2 DM is usually the type of diabetes that is diagnosed in patients older than 30 years of age, but it also occurs in children and adolescents. Clinically, it is characterized by hyperglycemia and insulin resistance. Type-2 DM is commonly associated with obesity, especially of the upper body (visceral/abdominal), and often occurs after weight gain.

Type-2 DM is a heterogeneous group of disorders in which hyperglycemia results from both an impaired insulin secretory response to glucose and a decreased insulin effectiveness in stimulating glucose uptake by skeletal muscle and in restraining hepatic glucose production (insulin resistance). The resulting hyperglycemia may lead to other common conditions, such as obesity, hypertension, hyperlipidemia, and coronary artery disease.

TSH can be administered to an individual at dosages described below. TSH can also be administered in conjunction with insulin, and other anti-diabetic drugs such as tolbutamide, chlorpropamide, etc.

Formulations and Administration of TSH

TSH can be administered to a human patient, alone or in pharmaceutical compositions where it is mixed with suitable carriers or excipient(s) at therapeutically effective doses to treat or ameliorate diseases associated with obesity and diabetes. Treatment dosages of TSH should be titrated to optimize safety and efficacy. Methods for administration include intravenous, intraperitoneal, rectal, intranasal, pulmonary, subcutaneous, and intramuscular. Pharmaceutically acceptable carriers will include water, saline, and buffers, to name just a few. Dosage ranges would ordinarily be expected to be from 0.1 kg to 1 mg per kilogram of body weight per day. A useful dose to try initially would be 25 μg/kg per day. However, the doses may be higher or lower as can be determined by a medical doctor with ordinary skill in the art. For a complete discussion of drug formulations and dosage ranges see Reminigton's Pharmaceutical Sciences, 17th Ed., (Mack Publishing Co., Easton, Penn., 1990), and Goodman and Gilman's: The Pharmacological Basis of Therapeutics, 9th Ed. (Pergamon Press 1996).

For pharmaceutical use, the proteins of the present invention can be administered orally, rectally, parenterally (particularly intravenous or subcutaneous), intracisternally, intravaginally, intraperitoneally, topically (as powders, ointments, drops or transdermal patch) bucally, or as a pulmonary or nasal inhalant. Intravenous administration will be by bolus injection or infusion over a typical period of one to several hours. In general, pharmaceutical formulations will include a TSH protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Doses of TSH polypeptide will generally be administered on a daily to weekly schedule. Determination of dose is within the level of ordinary skill in the art. The proteins may be administered for acute or chronic treatment, over several days to several months or years. In general, a therapeutically effective amount of TSH is an amount sufficient to produce a clinically significant decrease in weight, improvement in the diabetic state associated with obesity, decrease in liver steatosis, and/or increase in insulin sensitivity.

The invention is further illustrated by the following non-limiting examples.

### EXAMPLE 1

### TSH Activation of 3T3 L1 Adipocytes and Human Adipocytes Results in cAMP Production

Summary

Differentiated murine 3T3 L1 adipocytes and primary human adipocytes were used to study signal transduction of TSH. 3T3 L1 fibroblasts were differentiated into adipocytes and the cells were transduced with recombinant adenovirus containing a reporter construct, a firefly luciferase gene under the control of cAMP response element (CRE) enhancer sequences. This assay system detects cAMP-mediated gene induction downstream of activation of Gₛ-coupled G-protein coupled receptors (GPCR's). Treatment of the differentiated 3T3 L1 cells with isoproterenol, a β-adrenoreceptor agonist, resulted in elevation of cAMP levels and a 50-fold induction of luciferase expression. Treatment of differentiated 3T3 L1 cells with TSH also resulted in elevated cAMP levels and a 24-fold induction of luciferase expression. In a separate experiment, undifferentiated 3T3 L1 fibroblasts were transduced with the recombinant adenovirus. Treatment of the fibroblasts with TSH did not result in an increase in reporter gene induction. In another experiment, human primary adipocytes were also transduced with the recombinant adenovirus containing a reporter construct. Treatment of the human adipocytes with isoproterenol produced a 22-fold induction of luciferase expression. Treatment of the human adipocytes with TSH resulted in a 20-fold induction of the reporter gene. These results demonstrate TSH signaling through a GPCR in murine adipocytes and human adipocytes, and the production of cAMP levels similar to those achieved through β-adrenoreceptor stimulation.

Experimental Procedure

3T3 L1 cells were obtained from the ATCC (CL-173, Manasas, Va.) and cultured in growth medium as follows: the cells were propagated in DMEM high glucose (Life Technologies, cat. # 11965-092) containing 10% bovine calf serum (JRH Biosciences, cat. # 12133-78P). Cells were cultured at 37° C. in an 8% CO₂ humidified incubator. Cells were seeded in collagen-coated 96-well plates (Becton Dickinson, cat. # 356407) at a density of 5,000 cells per well. Two days later, differentiation medium was added as follows: DMEM high glucose containing 10% fetal bovine serum (Hyclone, cat. # SH30071), 1 μg/ml insulin, 1 μM dexamethasone, and 0.5 mM 3-isobutyl-methyl xanthine (ICN, cat. #195262). The cells were incubated at 37° C. in 8% CO₂ for 4 days and the medium replaced with DMEM high glucose containing 10% fetal bovine serum and 1 μg/ml insulin. The cells were incubated at 37° C. in 8% CO₂ for 3 days, then the medium was replaced with DMEM high glucose containing 10% fetal bovine serum. The cells were incubated at 37° C. in 8% CO₂ for 3 days, and the medium was replaced with DMEM low glucose (Life Technologies, cat. # 12387-015) containing 10% fetal bovine serum. The day before the assay, the cells were rinsed with F12 Ham (Life Technologies, cat. # 12396-016) containing 2 mM L-glutamine (Life Technologies, cat. # 25030-149), 0.5% bovine albumin fraction V (Life Technologies, cat. # 15260-037), 1 mM MEM sodium pyruvate (Life Technologies, cat. # 11360-070), and 20 mM HEPES. Cells were transduced with AV KZ55, an adenovirus vector containing KZ55, a CRE-driven luciferase reporter cassette, at 5,000 particles per cell. Following overnight incubation, the cells were rinsed once with assay medium (F12 HAM containing 0.5% bovine albumin fraction V, 2 mM L-glutamine, 1 mM sodium pyruvate, and 20 mM HEPES). 50 μl of assay medium were added to each well followed by 50 μl of 2× concentrated test protein. The plate was incubated at 37° C. in 5% CO₂ for 4 hours. Medium was removed from the plate and the cells were lysed with 25 μl per well of IX cell culture lysis reagent supplied in a luciferase assay kit (Promega, cat. # E4530). The cells were incubated at room temperature for 15 minutes. Luciferase activity was measured on a microplate luminometer (Perkin Elmer Life Sciences, Inc., model LB 96V2R) following automated injection of 40 μl of luciferase assay substrate into each well. The method described above, with modifications, was also used to test TSH and isoproterenol on human adipocytes obtained from Stratagene (cat. # 937236) seeded in 96-well plates. Human adipocytes were rinsed once with basal medium (Stratagene, cat. # 220002) containing 0.5% bovine albumin fraction V, then transduced with AV KZ55 at 5,000 particles per cell. Following overnight incubation, the cells were rinsed once with assay medium comprised of basal medium containing 0.5% bovine albumin fraction V and assayed as described above.

### EXAMPLE 2

### TSH-Induced Lipolysis in 3T3 L1 Adipocytes

Summary

3T3 L1 adipocytes were treated with TSH and the non-specific β-adrenoreceptor agonist isoproterenol for 4 hours. Lipolysis was assessed by the accumulation of glycerol and FFAs in the conditioned medium. FIG. 1 displays dose-response curves of TSH and isoproterenol for glycerol (upper panel) and FFA (lower panel). TSH potently stimulated lipolysis in the murine adipocytes, as shown in FIG. 1.

Measurement of Free Fatty Acids in Conditioned Media From Differentiated 3T3 L1 Cells

Free fatty acids were measured using the Wako NEFA C kit (Wako Chemicals GmbH, Neuss, Germany) for quantitative determination of non-esterified (or free) fatty acids with a modified protocol. Isoproterenol (ICN), a lipolysis-inducing positive control, was diluted to a starting concentration of 2 μM in assay medium (Life Technologies low glucose DMEM, 1 mM sodium pyruvate, 2 mM L-glutamine, 20 mM HEPES, and 0.5% BSA). The isoproterenol was further diluted in half log serial dilutions. TSH was serially diluted down to 0.06 nM. The medium was removed from 3T3 L1 adipocytes in 96-well plates. 50 μl of assay medium were added to each well, followed by 50 μl of TSH or isoproterenol to each well. The plates were incubated for 4 hours at 37° C. 40 μl of conditioned medium were collected for glycerol assay analysis, and 40 μl of conditioned medium were collected for free fatty acid analysis. Oleic acid (Sigma) was dissolved in methanol and used as a reference for determining the amount of free fatty acids in the conditioned media. Wako reagents A and B were reconstituted to 4× the recommended concentration. Conditioned media samples were assayed in 96-well plates. 50 μl of Wako reagent A were added to 5 μl of oleic acid standard plus 40 μl of assay medium. 50 μl of Wako reagent A were added to 40 μl of conditioned medium from differentiated 3T3 L1 cells and 5 μl of methanol. The 96-well plates were incubated at 37° C. for 10 minutes. 100 μl of Wako reagent B were added to each well. The 96-well plates were incubated at 37° C. for 10 minutes. The 96-well plates were then allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

Measurement of Glycerol in Conditioned Media from Differentiated 3T3 L1 Cells

Glycerol was measured in conditioned media using the Sigma Triglyceride (GPO-Trinder) kit with a modified protocol. Isoproterenol was diluted to a starting concentration of 2 μM. The isoproterenol was further diluted in half log serial dilutions. TSH was diluted to starting concentrations of 300 nM in assay medium. TSH was then serially diluted down to 0.06 nM. Medium was removed from 3T3 L1 adipocytes in 96-well plates. 50 μl of assay medium were added to each well, followed by 50 μml of TSH or isoproterenol to each well. The plates were incubated for 4 hours at 37° C. 40 μl of conditioned medium were collected for glycerol assay analysis, and 40 μl of conditioned medium were collected for free fatty acid analysis. The glycerol standard was diluted in water to a range from 200 nmols/10 μl to 0.25 nmols/10 μl. Glycerol was used as a reference for determining the amount of glycerol in the conditioned media. Sigma reagent A was reconstituted to the recommended concentration. Conditioned media samples were assayed in 96-well plates. 150 μl of Sigma reagent A were added to 10 μl of glycerol standard plus 40 μl of assay medium. 150 μl of Sigma reagent A were added to 40 μl of conditioned medium from differentiated 3T3 L1 cells plus 10 μl of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 3

### Stimulation of Lipolysis by TSH In Vivo

Summary

TSH, the β₃-adrenoreceptor agonist CL 316,243 (CL), and saline vehicle were examined for stimulation of lipolysis in mice following an overnight fast. Mice (n=7-8/group) were bled immediately before IP injection of TSH (300 μg/kg), CL (1 mg/kg), or vehicle, and blood sampled by retro orbital draws at 2 and 4 hours post-injection. Lipolysis was assessed as the change in serum glycerol or FFA compared to baseline. FIG. 2 shows the changes in levels of glycerol (Lg/ml in serum, upper panel) and FFA (μg/ml in serum, lower panel) for the treatment groups. TSH administration stimulated increased serum glycerol levels at 2 and 4 hours, compared to the vehicle controls (148+/−23, (p=. 09) and 165+/−15 μg/ml (p<. 001) versus 80+/−29 and −62+/−14). TSH increased serum FFA 1,477+/−219 (p<. 001) and 1506+/−94 (p<. 001) at 2 and 4 hours compared to vehicle values −20+/−77 and ˜466+/−67, respectively (all errors are standard error of the mean). The control β-AR agonist also significantly elevated serum glycerol and FFA levels.

Treatment Protocol

C57BL/6 male ob/ob mice, age 10 weeks, were grouped to normalize weight (n=7-8 for each treatment; average group weight=37.8 g+/−0.4 g). Mice were housed individually for 18 hours prior to treatment, at which time food was withdrawn, with free access to water given. At approximately 8 a.m., the subjects were anesthetized with halothane and blood samples taken by retro-orbital eye bleeds. The blood was allowed to clot, and the serum was separated by centrifugation and frozen for later analysis. Test substances were administered by IP injection in a volume of 0.1 ml, and the animals replaced in their cages for 2 hours with free access to water. At 2 hours, the mice were sacrificed and blood drawn by cardiac puncture.

Measurement of Glycerol and FFA in Murine Serum

For measuring free fatty acids in serum, the method previously described for measuring free fatty acids in conditioned medium was followed, with the following modifications. Wako reagents A and B were reconstituted to 2× the recommended concentration. 75 μl of Wako reagent A were added to 5 μl of oleic acid standard plus 5 μl of water. 75 μl of Wako reagent A were added to 5 μl of serum plus 5 μl of methanol (to mirror the oleic acid standard conditions). The 96-well plates were incubated at 37° C. for 10 minutes. 150 μl of Wako reagent B were added to each well. The 96-well plates were incubated at 37° C. for 10 minutes. The 96-well plates were allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

For measuring glycerol in serum, the method previously described for measuring glycerol in conditioned medium was followed, with the modifications described below.

Sigma reagent A was reconstituted to 0.5× the recommended concentration. 200 μl of Sigma reagent A were added to 10 μl of glycerol standard. 200 μl of Sigma reagent A were added to 5 μl of serum plus 5 μl of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 4

### Chronic Treatment of ob/ob Mice with TSH

### SUMMARY

TSH was administered daily for 28 days to obese male ob/ob mice. Data was obtained for weight, food intake, glucose, insulin, lipid and thyroid hormone. A subset of the animal groups was subjected to a glucose tolerance test at the end of the study. At sacrifice, animals were examined for changes in adipose depot weights, liver pathology, and gross histology. As described below, TSH treatment resulted in decreased resting glucose and insulin levels, and increased insulin sensitivity in a glucose tolerance test. Serum triglyceride and cholesterol levels were significantly reduced compared to controls, and thyroid hormone levels were not elevated above the vehicle group. Necropsy analysis of adipose tissues revealed substantial and significant increases in inter-scapular brown adipose tissue (IBAT), and significant decreases in intra-abdominal mesangial white fat. The TSH treatment group showed a strong trend toward decreased weight gain compared to controls and thyroxine-treated animals. Evaluation of liver histology sections was performed to examine the effect of TSH-mediated lipolysis on liver steatosis. Prominent liver steatosis typically associated with the ob/ob strain employed in these studies was significantly reversed by TSH treatment, exhibiting marked reduction in fat deposition in liver hepatocytes. Thyroid hormone did produce a change in the extent of steatosis.

Treatment protocol

11-week old male ob/ob mice were individually caged and given a standard lab chow (4% fat) with free access to food and water. Animals were assigned to a treatment group (n=7-8, average weight 54.3+/−0.3 g per group), kept on a 12 hour dark cycle (6 PM to 6 AM), and injected each day between 7 and 9 AM. Chow consumed by each animal was weighed twice weekly. All animals received treatments IP with an injection volume of 0.1 ml. TSH was administered at 267 μg/kg, and the H₃-AR agonist CL 316,243 at 0.75 mg/kg. Thyroid hormone (T₄) was administered at 1.5 μg/mouse for 4 days, reduced to 1 [μg/mouse for 10 days, and returned to 1.5 μg/mouse for the next 14 days. The vehicle controls received sterile saline. TSH was obtained from Genzyme Pharmaceuticals, (Thyrogen®, catalog number 36778; Genzyme Corporation, Cambridge, Mass.), CL316,243 from Sigma Biochemicals, and T₄ obtained from Calbiochem, Inc. All blood draws were performed by retro-orbital puncture under isoflurane anesthesia.

Body Weight and Food Intake

Food intake did not differ significantly between groups (vehicle 5.9+/−0.22, CL16,243 6.3+/−0.11, TSH 5.9+/−0.36, and thyroxine 6.1+/−0.17 grams/day of chow). Body weight changes were assessed as the percentage increase in body weight from the beginning of the study. The weight in the vehicle group increased 8.8+/−0.6%. The thyroxine group had a slightly greater increase in weight (9.4+/−0.5%), and the β₃-AR group a slightly lower increase in weight (7.7+/−41%) compared to the vehicle controls. The TSH-treated group showed less weight gain than the vehicle controls (4.6+/−2.4%, p=0.11), with 3 of the 8 members of the group demonstrating an overall decrease in weight, the only animals in the study to do so.

Measurement of Serum Thyroxine Levels

After 25 days of treatment as described above, blood was sampled from all treated animals (n=7-8/group), serum separated, and analyzed for total T₄ by a commercially available kit (Biocheck, Burlingame, Calif.). FIG. 3 shows the levels of thyroxine determined for each group +/−standard error. After 25 days of treatment, the vehicle T₄ levels were 5.14+/−0.08 μg/dl. The TSH-treated group had T₄ levels of 5.31+/−0.16, the β₃-AR receptor group 5.14+/−0.19, and the thyroxine-treated group 9.04+/−0.47 μg/dl. The β₃-AR and TSH-treated groups had circulating levels of T₄ that were significantly lower than controls (p<0.02) and the thyroxine treatment group had levels significantly higher than vehicle controls (p<0.001).

Adipose Tissue Analysis

Four animals from each group were analyzed for evaluation of changes in adipose tissue depot mass after 4 weeks of treatment. Following sacrifice, brown scapular fat (IBAT) and intra-abdominal fat were dissected and the tissues weighed. All 3 treatment groups showed significant increases in brown scapular fat mass. Thyroxine showed the largest increase (1.26+/−0.03 g, p<0.001) vs. the vehicle control (0.66+/−0.07 g). Elevated thyroid hormone levels are known to act to stimulate]BAT. TSH and CL 316,243 also increased IBAT mass (1.25+/−0.13, p<0.01 and 0.87+/−0.03, p<0.05, respectively). These increases in IBAT are associated with increased metabolic rate as described above. The mesangial fat depot was dissected and removed from the colon for weight determination. Mesangial fat is white adipose tissue and is only readily visualized and dissected in obese mice. Mesangial white fat was removed by carefully stripping the fat, associated matrix and vascular supporting bed from the length of the colon. The weight of the fat and matrix removed from the vehicle controls was 1.31+/−0.04 g, and the material removed was quite white in appearance from the fat cells in the removed mass. The weights of depots removed from the CL 316,243, TSH, and T₄ treatment groups were 1.53+/−0.10 g (p=0.08), 1.11+/−0.03 g (p<0.02), and 1.18+/−0.11 g (p=0.32), respectively. The appearance of the mesangial depots removed from the thyroxine and particularly the TSH was much less white due to decreased fat content in the depot, and suggested that the relative loss in fat cell mass within the depot was larger than the change in weight of the removed structure suggested.

Liver Steatosis

Liver sections were dissected from all treatment groups described above and mounted in paraffin following fixation with NBS-formalin. Sections were mounted and stained with hematotoxylin and eosin (H&E) for visualization of hepatic structural changes. The extent of liver steatosis was evaluated on a four-point scale, from 0 to 3, with zero displaying no signs of liver steatotosis, and 4, representing pronounced macrovesicular and microvesicular steatosis. The averages of the groups (n=4) showed significant differences in the extent of steatosis as judged by the size of the lipid inclusions and the integrity of the hepatocyte structure visible in the sections. Average scores given to the groups were vehicle (4), thyroxine (3), TSH (2), and CL 316,243 (1).

IPGTT

Following 4 weeks of daily treatment as described above, mice (n=4/group) were fasted for four hours immediately following the beginning of the light cycle, and a blood sample obtained before IP injection of a glucose solution (1.5 g/kg body weight). Blood samples were obtained at 20, 40, and 120 minutes following injection to evaluate changes in serum glucose and insulin. Glucose concentrations were determined with a Freestyle blood glucose monitor (Therasense Corp.), and insulin concentrations were determined with a commercial ELISA kit (Alpco Diagnostics, Windham N. H.). As shown in FIG. 4, baseline blood glucose levels were significantly lower in the CL-treated animals (139+/−9, p<0.05) and the TSH-treated animals (114+/−7, p=0.02), than in the vehicle controls (218+/−25 mg/dl). The thyroxine-treated animals were not significantly different from the vehicle controls (252+/−34). As shown in FIG. 5, the control P₃-AR agonist and TSH-treated groups exhibited increased insulin sensitivity and increased clearance of the glucose load administered at time zero compared to vehicle controls. In particular, the thyroxine-treated group did not exhibit increased glucose clearance compared to vehicle-treated controls, providing evidence that the TSH-mediated effects are not mediated via the thyroid axis, but through the stimulation of lipolysis (serum glucose of TSH-treated and thyroxine-treated groups at 120 minutes post glucose injection had values of 361+/−37 and 802+/−69 mg/dl, respectively, p<0.005).

Serum Lipid Analysis

The study set used for the IPGTT was treated an additional 7 days before sacrifice (total treatment time of 5 weeks). Subject animals were fasted for 4 hours at the beginning of the light cycle, and serum was obtained at sacrifice under isoflurane anesthesia. Triglyceride and total cholesterol levels were determined with the Cholestech LDX blood analyzer (Cholestech Corporation, Hayward Calif.). Serum triglyceride levels for the vehicle controls and P₃-AR agonist CL 316,243 were 164+/−34 and 191+/−9 mg/dl, respectively. The serum triglycerides in the TSH-treated group were lower (80+/−10 mg/dl, p=0.05), and the triglycerides in the thyroxine treated group higher than the vehicle controls (297+/−30 mg/dl, p=0.05). Total cholesterol levels in the vehicle-treated, β₃-AR agonist-treated, and thyroxine-treated groups were 220+/−16, 198+/−7, and 231+/−11 mg/dl, respectively. Total cholesterol in the TSH treatment group was significantly lower at 124+/−16 mg/dl, p<0.001.

We claim:

1. A method for inducing lipolysis in a mammal comprising administering to the mammal a pharmaceutically effective amount of a TSH polypeptide, wherein administration of the polypeptide results in a clinically significant decrease in the body weight of the mammal.

2. The method of claim 1, wherein said mammal is obese.

3. The method of claim 1, wherein said mammal has a body mass index greater than 25.

4. The method of claim 3, wherein said body mass index is 26, between 26 and 50, or 50.

5. The method of claim 1, whereby the decrease in body weight of the mammal results from lipolytic stimulation of adipose tissue.

6. A method for inducing weight loss in a mammal, comprising administering to the mammal a pharmaceutically effective amount of a TSH polypeptide, wherein administration of the polypeptide results in a clinically significant decrease in body weight of the mammal.

7. The method of claim 5, wherein said mammal is obese.

8. The method of claim 6, wherein said mammal has a body mass index greater than 25.

9. The method of claim 7, wherein said body mass index is 26, between 26 and 50, or 50.

10. A method for treating type-2 diabetes in a mammal, comprising administering to the mammal a pharmaceutically effective amount of a TSH polypeptide, wherein administration of the polypeptide results in an improvement in the diabetic state of the mammal.

11. The method of claim 10, wherein said mammal is obese.

12. A method for treating hyperlipidemia in a mammal comprising administering to the mammal a pharmaceutically effective amount of a TSH polypeptide, wherein administration of the polypeptide results in decreased hyperlipidemia in the mammal.

13. The method of claim 12, wherein said mammal is obese.

14. The method of claim 12, wherein said mammal is type-2 diabetic.

15. The method of claim 12, wherein administration of the polypeptide results in decreased serum glucose or insulin levels.

16. A method for treating steatohepatitis in a mammal, comprising administering to the mammal a pharmaceutically effective amount of a TSH polypeptide, wherein administration of the polypeptide results in an improved steatohepatic state in the mammal.

17. The method of claim 16, wherein said mammal is obese.

18. The method of claim 16, wherein said mammal is type-2 diabetic.

19. The method of claim 16, wherein serum cholesterol or serum triglyceride levels are decreased.

20. A method for preventing steatohepatitis in a mammal with steatosis, comprising administering to the mammal a pharmaceutically effective amount of a TSH polypeptide, wherein administration of the polypeptide maintains or reduces the steatosis.

21. The method of claim 20, wherein said mammal is obese.

22. The method of claim 20, wherein said mammal is type-2 diabetic.

23. A method for lowering elevated plasma cholesterol levels in a mammal, comprising administering a pharmaceutically effective amount of a TSH polypeptide to said mammal, wherein administration of the polypeptide lowers the plasma cholesterol level in the mammal.

24. The method of claim 23, wherein the mammal is hypercholexterolemic.

25. The method of claim 23, wherein the mammal is type-2 diabetic.

26. A method of lowering elevated triglyceride levels in a mammal, comprising administering a pharmaceutically effective amount of a TSH polypeptide to said mammal, wherein administration of the polypeptide lowers triglyceride levels in the mammal.

27. The method of claim 26, wherein the mammal is type-2 diabetic.

28. A method for treating steatosis of the liver in a mammal with steatosis, comprising administering to the mammal a pharmaceutically effective amount of a TSH polypeptide, wherein administration of the polypeptide maintains or reduces the steatosis.

29. The method of claim 28, wherein the mammal is obese.

30. The method of claim 28, wherein the mammal is type-2 diabetic.

31. A method for improving insulin sensitivity in a mammal comprising administering to the mammal a pharmaceutically effective amount of a TSH polypeptide, wherein administration of the polypeptide results in increased sensitivity to insulin.

32. The method of claim 31, wherein blood glucose levels are decreased.

33. The method of claim 31, wherein insulin levels are decreased.

34. A method for treating athersclerosis in a mammal comprising administering to the mammal a pharmaceutically effective amount of a TSH polypeptide, wherein administration of the polypeptide results in an improved athersclerotic state.

35. The method of claim 34, wherein the mammal is hyperlipidemic.


SEQUENCE LISTING (6 sequences)

SEQ ID NO: 1 - DNA, 351 bp, Homo sapiens
  CDS (1)...(351)
atggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 2 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - protein, 92 aa, Homo sapeins
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 4 - DNA, 417 bp, Homo sapiens
  CDS (1)...(417)
atgactgctctctttctgatgtccatgctttttggccttgcatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaa

SEQ ID NO: 5 - protein, 138 aa, Homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 6 - protein, 112 aa, Homo sapiens
FCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSY